(19) United States
(12) Patent Application Publication
(10) Pub. No.: US 2024/0398952 A1
(43) Pub. Date: Dec. 5, 2024

(54) PLASMA ACTIVATED PRODUCT

(71) Applicant: Tahereh Safari, Qom (IR)

(72) Inventor: Tahereh Safari, Qom (IR)

(21) Appl. No.: 18/676,324

(22) Filed: May 28, 2024

Related U.S. Application Data
(60) Provisional application No. 63/469,508, filed on May 29, 2023.

(51) Int. Cl. A61K 41/00; H05H 1/24

(52) U.S. Cl. CPC A61K 41/00; H05H 1/247; H05H 2245/20; H05H 2245/30

(57) ABSTRACT

In accordance with some embodiments herein, a plasma production system is provided. The plasma production system includes a bioreactor tank housing: an electrolyte mixture including sodium chloride (NaCl) and a liquid; a first electrode at least partially surrounded by the electrolyte mixture; and a second electrode at least partially surrounded by the electrolyte mixture. The plasma production system includes a power supply electrically connected to the first electrode and the second electrode. The power supply is configured to supply electrical power to the electrolyte mixture via the first electrode and the second electrode to produce a plasma activated product.

### RELATED APPLICATION

This application claims the benefit of U.S. Provisional Patent Application No. 63/469,508, filed May 29, 2023, which is incorporated herein by reference in its entirety.

### BACKGROUND

Cancer is a significant health challenge in the world today. Different types of cancer may be distinguished from each other based upon the location and manner of growth of abnormal cells in different parts of a patient's body. Types of cancer include carcinoma, sarcoma, leukemia, lymphoma, myeloma, etc.

### DESCRIPTION OF THE DRAWINGS

While the techniques presented herein may be embodied in alternative forms, the particular embodiments illustrated in the drawings are only a few examples that are supplemental of the description provided herein. These embodiments are not to be interpreted in a limiting manner, such as limiting the claims appended hereto.

FIG. 1A presents an illustration of a bioreactor tank, according to some embodiments.

FIG. 1B presents an illustration of a bioreactor tank housing a liquid, according to some embodiments.

FIG. 1C presents an illustration of adding sodium chloride (NaCl) to a liquid within a bioreactor tank, according to some embodiments.

FIG. 1D presents an illustration of an electrolyte mixture within a bioreactor tank, according to some embodiments.

FIG. 2A presents an illustration of an example plasma production system before operation, according to some embodiments.

FIG. 2B presents an illustration of an example plasma production system at first stage of operation, according to some embodiments.

FIG. 2C presents an illustration of an example plasma production system at second stage of operation, according to some embodiments.

FIG. 2D presents an illustration of an example plasma production system after the second stage of operation, according to some embodiments.

FIG. 2E presents an illustration of an example plasma production system after a third period of time, according to some embodiments.

FIG. 2F presents an illustration of a plasma activated water produced by a plasma production system, according to some embodiments.

FIG. 2G presents an illustration of a plasma activated sediment produced by a plasma production system, according to some embodiments.

FIG. 3 presents an illustration of an example plasma production system utilizing a plurality of electrodes, according to some embodiments.

FIG. 4 presents an illustration of an example plasma production system utilizing an electrode as an anode and a coil-shaped electrode as a cathode, according to some embodiments.

FIG. 5 presents an illustration of an example plasma production system utilizing an electrode as a cathode and a coil-shaped electrode as an anode, according to some embodiments.

FIG. 6 presents an illustration of an example plasma production system utilizing a first coil-shaped electrode as a cathode and a second coil-shaped electrode as an anode, according to some embodiments.

FIG. 7 is a flow diagram illustrating a method of producing a plasma activated product, according to some embodiments.

FIG. 8 presents a chart of cancer cell lethality on MCF7 (Breast Cancer Cell Line) corresponding to anti-cancer drugs, according to some embodiments.

FIG. 9 presents a table of animal testing parameters associated with treatment effectiveness of a plasma activated product on mice corresponding to different types of administration of the plasma activated product, according to some embodiments.

FIG. 10A presents an illustration of positron emission tomography (PET) scan corresponding to a cancer patient, according to some embodiments.

FIG. 10B presents an illustration of a first computed tomography (CT) scan corresponding to a cancer patient before plasma activated product treatment, according to some embodiments.

FIG. 10C presents an illustration of a second computed tomography (CT) scan corresponding to a cancer patient after plasma activated product treatment, according to some embodiments.

FIG. 10D presents a table of human testing report associated with tumor marker parameters before and after plasma activated product treatment corresponding to a cancer patient, according to some embodiments.

### DETAILED DESCRIPTION

The following subject matter may be embodied in a variety of different forms, such as methods, compositions, materials, and/or systems. Accordingly, this subject matter is not intended to be construed as limited to any example embodiments set forth herein. Rather, example embodiments are provided merely to be illustrative.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. Ranges from any lower limit to any upper limit are contemplated. The upper and lower limits of these smaller ranges which may independently be included in the smaller ranges is also encompassed within the disclosure, subject to any specifically excluded limit in the stated range. Where the stated range includes one or both of the limits, ranges excluding either both of those included limits are also included in the disclosure.

Although any methods and materials similar or equivalent to those described herein can also be used in the practice or testing of the present disclosure, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure. All publications, patent applications, patents, figures and other references mentioned herein are expressly incorporated by reference in their entirety.

The present disclosure provides a plasma production system. In some examples, the plasma production system comprises a bioreactor tank. The plasma production system includes a bioreactor tank housing an electrolyte mixture comprising sodium chloride (NaCl) and/or a liquid. The plasma production system includes a first electrode at least partially surrounded by the electrolyte mixture. The plasma production system includes a second electrode at least partially surrounded by the electrolyte mixture. The plasma production system includes a power supply electrically connected to the first electrode and/or the second electrode. The power supply is configured to supply electrical power to the electrolyte mixture via the first electrode and/or the second electrode to produce a plasma activated product.

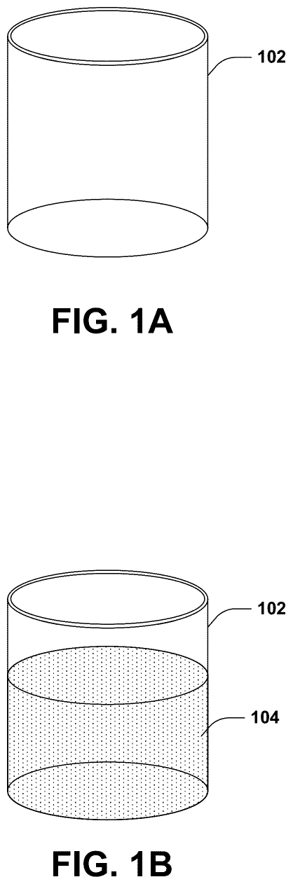

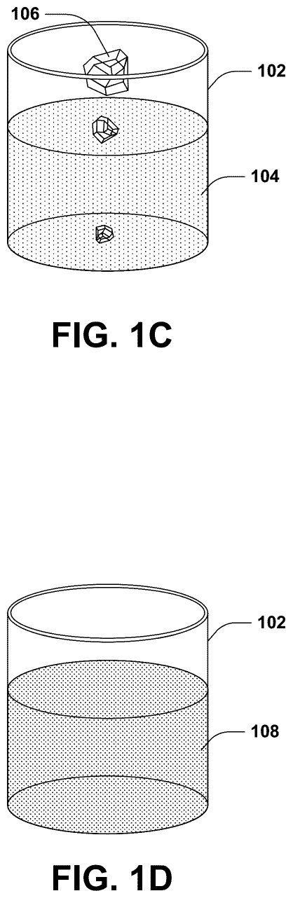

FIG. 1A illustrates an example of a bioreactor tank 102. The bioreactor tank 102 may comprise a container (e.g., an open top container) in which the container may be utilized for chemical processes. The bioreactor tank 102 may comprise one or more non-conductive materials (e.g., glass, plastic, wood, etc.). In some examples, the bioreactor tank 102 may be disinfected, sterilized and/or free from acidic and/or alkaline environment.

FIG. 1B, illustrates the bioreactor tank 102 housing a liquid 104. The liquid 104 may comprise water (e.g., distilled water, etc.). In some examples, to achieve higher amount of pureness, the liquid 104 may be distilled more than one time.

FIG. 1C, illustrates addition of mineral salt 106 (e.g., sodium chloride) to the liquid 104 within the bioreactor tank 102. In some examples, a ratio of the mineral salt 106 added to the liquid 104 by weight is between about 0.01:1 to about 0.2:1, such as between about 0.05:1 to about 0.1:1.

FIG. 1D illustrates an electrolyte mixture 108 which may be produced via mixing (e.g., dissolving) the mineral salt 106 with the liquid 104. In some examples, a ratio of the mineral salt 106 dissolved in the liquid 104 by weight is between about 0.01:1 to about 0.2:1, such as between about 0.05:1 to about 0.1:1. In some examples, the electrolyte mixture 108 may comprise ions (e.g., metal ions, hydrogen ions H⁺, hydroxide OH⁻). In some examples, the electrolyte mixture 108 may comprise positively charge ions (e.g., cations). In some examples, the electrolyte mixture 108 may comprise negatively charge ions (e.g., anions). In some examples, the electrolyte mixture 108 is electrically conductive which can be utilized in plasma production systems.

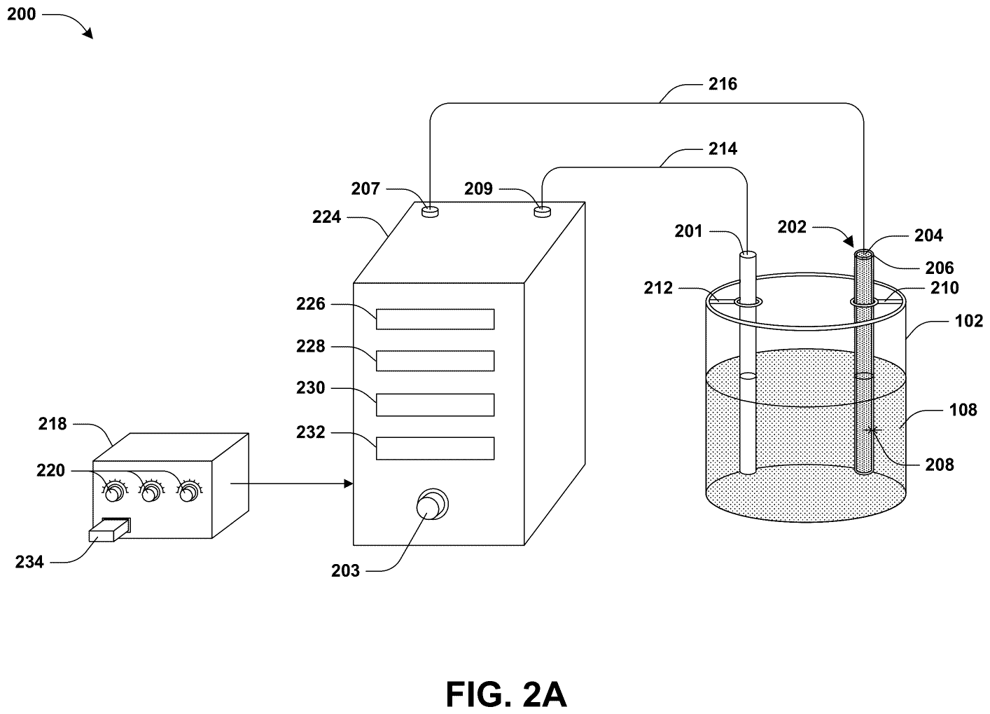

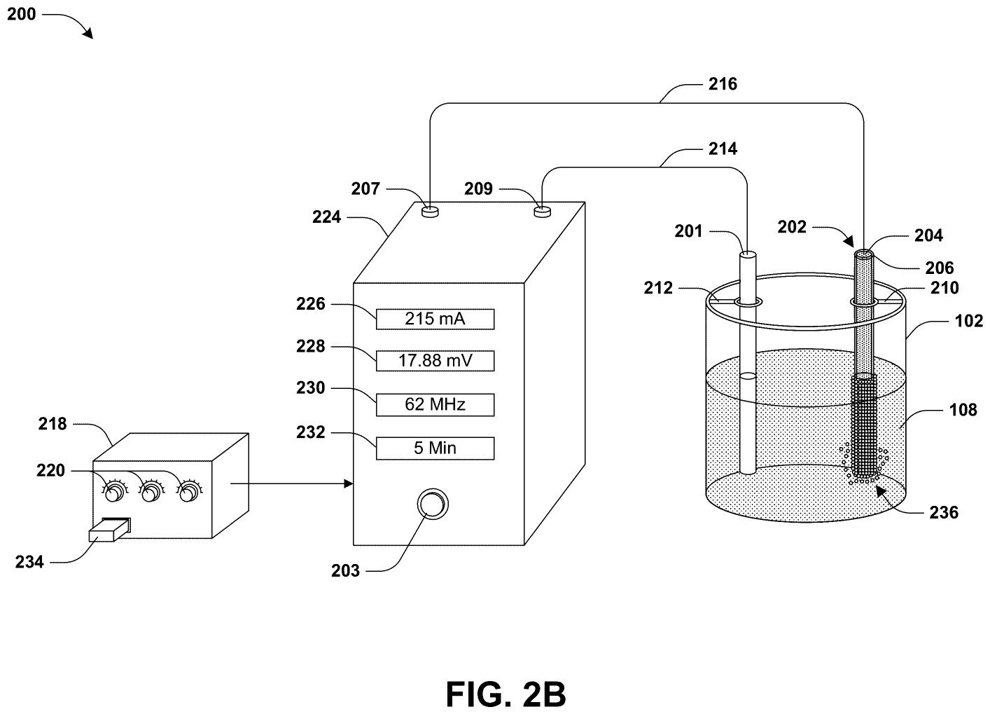

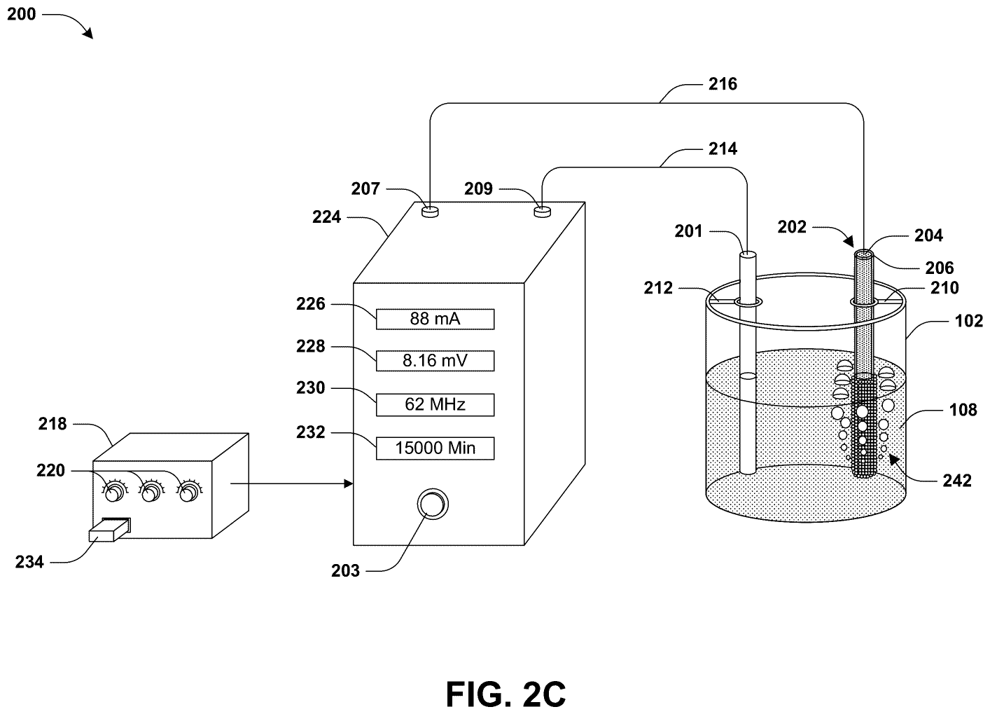

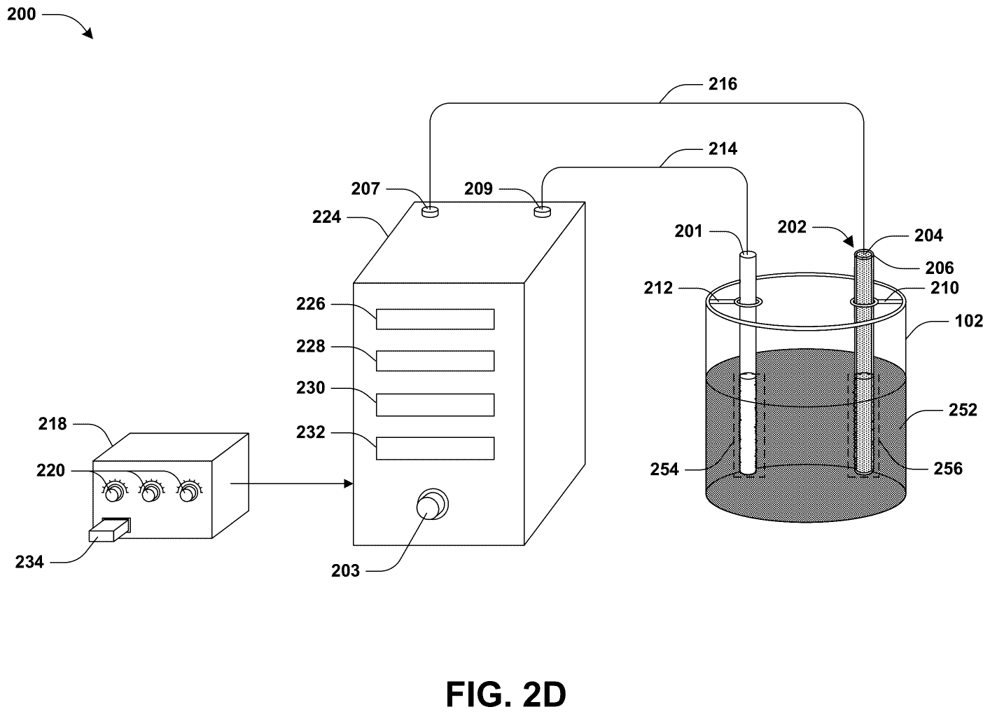

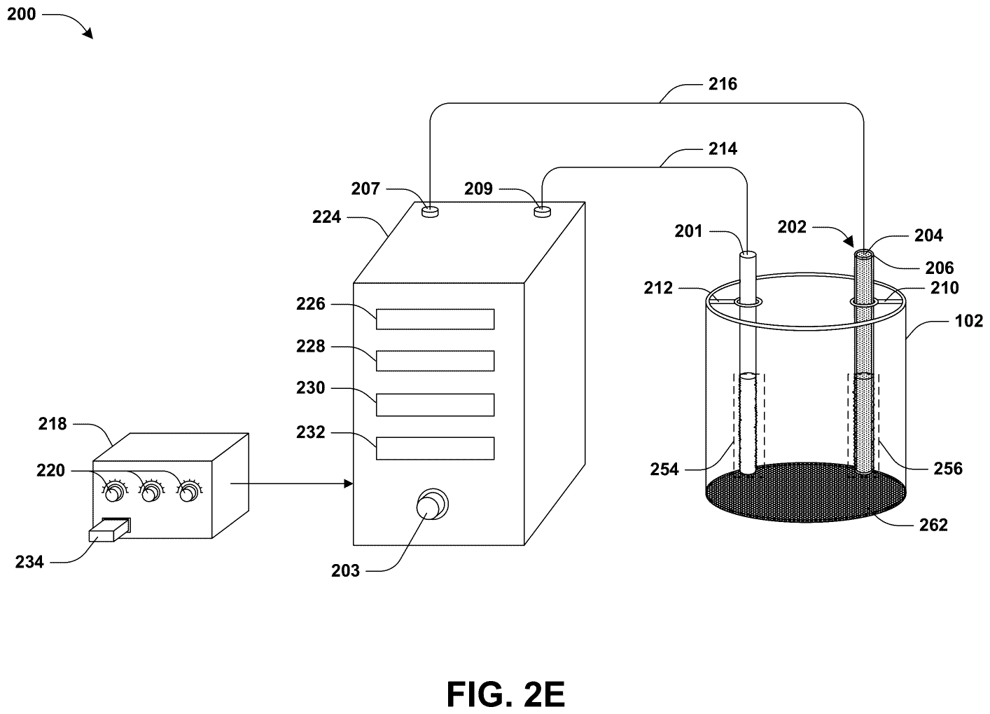

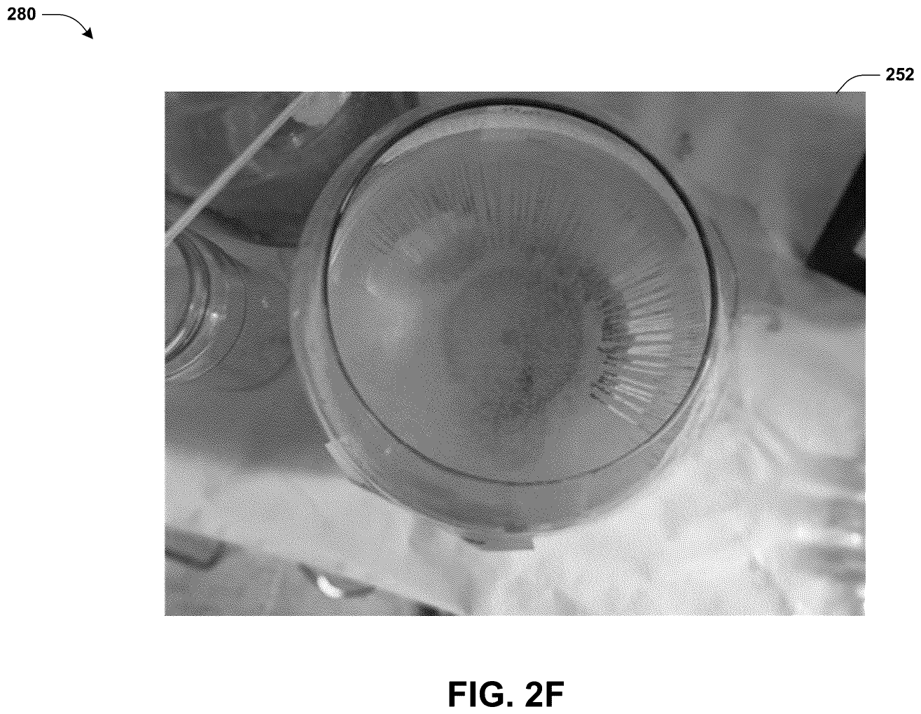

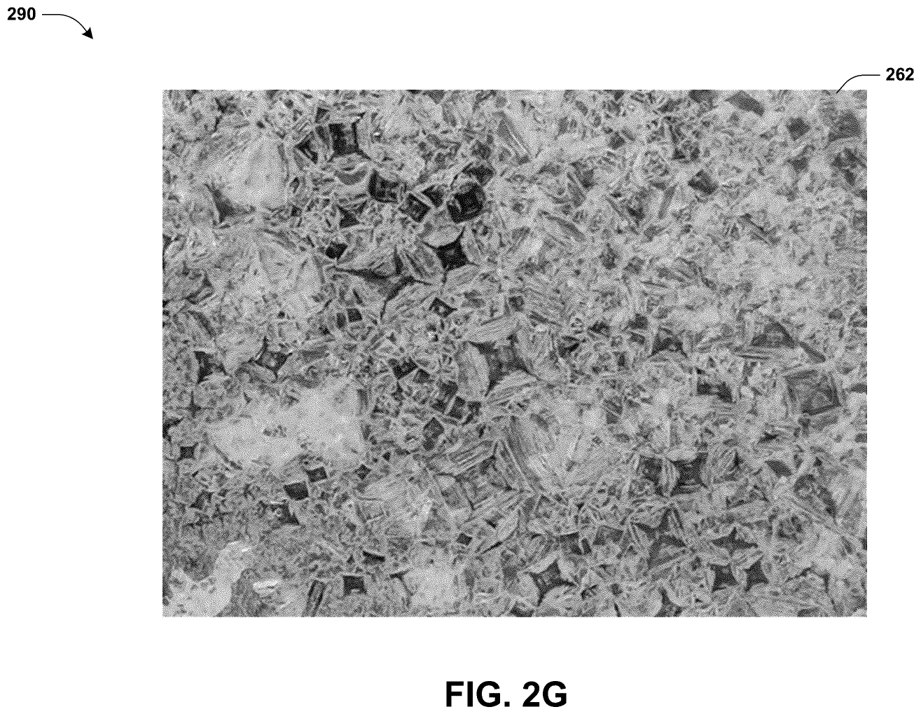

FIG. 2A-2E illustrates a plasma production system 200, in accordance with some embodiments. In some examples, the plasma production system 200 may comprise a bioreactor tank 102, a power supply 224 and/or a controller 218. FIG. 2A is an illustration of the plasma production system 200 before operation (e.g., when the power supply 224 is off and/or when the plasma production system 200 is inactive). In FIG. 2A, the bioreactor tank 102 may at least partially house an electrolyte mixture 108, a first electrode 201 and/or a second electrode 202, wherein the first electrode 201 and/or the second electrode 202 may be at least partially surrounded by the electrolyte mixture 108. In some examples, the first electrode 201 may be held and/or supported by a first holder 212, and/or the second electrode 202 may be held and/or supported by a second holder 210. In some examples, the first holder 212 and/or the second holder 210 may be attached to the bioreactor tank 102. In some examples, the first electrode 201 may comprise one or more metals (e.g., copper (Cu), gold (Au), silver (Ag), zinc (Zn) and/or iron (Fe)) which are electrically conductive. In some examples, the first electrode 201 may comprise one or more non-metals (e.g., graphite (Gr)) which are electrically conductive. In some examples, the second electrode 202 may comprise an electrode body 204 and/or a coating 206 wherein the coating 206 may cover at least a portion of the electrode body 204. The coating 206 may be at least partially surrounded by the electrolyte mixture 108. In some examples, the electrode body 204 may comprise one or more metals (e.g., copper (Cu), gold (Au), silver (Ag), zinc (Zn) and/or iron (Fe)) which are electrically conductive. In some examples, the electrode body 204 may comprise one or more non-metals (e.g., graphite (Gr)) which are electrically conductive. In some examples, the coating 206 may comprise a metal oxide layer and/or a graphite layer. In some examples, the electrode body 204 may be a copper body and/or the metal oxide layer may be a copper oxide layer. In some examples, the coating 206 may comprise a thickness 208. The thickness 208 is between about 1 nanometer to about 1000 nanometers, such as between about 50 nanometers to about 500 nanometers. Other values of the thickness 208 are within the scope of the present disclosure.

In some examples, the plasma production system 200 may comprise a power supply 224. The power supply 224 may be configured to supply electrical power to the electrolyte mixture 108 via the first electrode 201 and/or the second electrode 202 to produce a plasma activated product. In some examples, the plasma activated product may comprise plasma. In some examples, the plasma activated product may comprise a plasma activated liquid (e.g., a plasma activated water). In some examples, the plasma activated product may comprise a plasma activated sediment. In some examples, the plasma activated product may comprise a plasma activated gas. In some examples, the plasma activated product may be used as a medicine (e.g., a plasma activated medicine that may be biocompatible), such as for treatment of a patient. In some examples, the plasma activated product may be used as a fertilizer (e.g., a plasma activated fertilizer that may be biocompatible). Other suitable uses of the plasma activated product are within the scope of the present disclosure.

In some examples, the plasma activated product may comprise electromagnetic energy, biocompatible plasma waves, ionizing rays, free electrons, positive ions, negative ions, free radicals, gas atoms in basic states and/or gas atoms in excited states. In some examples, the plasma activated product may comprise constructive plasma waves and/or restorative plasma waves. In some examples, the plasma activated product may comprise plasma waves, ionization waves and/or ultraviolet waves.

In some examples, the plasma activated product may comprise a biocompatible plasma, wherein the biocompatible plasma may comprise electromagnetic waves. In some examples, the electromagnetic waves of the biocompatible plasma may comprise a frequency. In some examples the frequency of the electromagnetic waves of the biocompatible plasma may be between about 10 megahertz to about 100 megahertz, such as between about 50 megahertz to about 80 megahertz which may be in range of a human vibrational frequency (e.g., between about 62 megahertz to about 70 megahertz) associated with healthy and/or normal human cells. In some examples, the biocompatible plasma may be completely safe and/or the frequency of its electromagnetic waves may be in the range of the frequency of a body of a living being and the biocompatible plasma may be easily consumed (e.g., eaten). In some examples, intensity of the electromagnetic waves related to the biocompatible plasma is between about 0.5 milligauss (mG) to 10 milligauss, such as between about 1 milligauss to 3 milligauss. The intensity of electromagnetic waves related to the biocompatible plasma is near intensity of electromagnetic waves related to a human body.

In some examples, the plasma activated product may comprise physical waves. In some examples, the physical waves may comprise electromagnetic waves and/or plasma waves.

In some examples, the plasma activated product may comprise a cold atmospheric plasma, wherein the cold atmospheric plasma may be produced at ambient temperature (e.g., in a range from 59 degrees fahrenheit to 77 degrees fahrenheit) and/or atmospheric pressure. In some examples, the plasma activated product may comprise a non-thermal plasma.

In some examples, the power supply 224 may comprise a Direct Current (DC) power supply configured to supply DC electrical power to the electrolyte mixture 108. In some examples, the DC electrical power supplied by the power supply 224 may comprise a current that is between about 20 milliamperes to about 1000 milliamperes, such as between about 88 milliamperes to about 215 milliamperes. In some examples, the DC electrical power supplied by the power supply 224 may have a voltage that is between about 1 millivolt to about 100 millivolts, such as between about 8.16 millivolts to about 17.88 millivolts. In some examples, the power supply 224 may comprise an Alternating Current (AC) power supply configured to supply AC electrical power to the electrolyte mixture 108. In some examples, the AC electrical power supplied by the power supply 224 may comprise a current that is between about 20 milliamperes to about 1000 milliamperes, such as between about 88 milliamperes to about 215 milliamperes. In some examples, the AC electrical power supplied by the power supply 224 may comprise a voltage that is between about 1 millivolt to about 100 millivolts, such as between about 8.16 millivolts to about 17.88 millivolts. In some examples, the AC electrical power supplied by the power supply 224 may comprise a frequency that is between about 1 megahertz to about 1000 megahertz, such as between about 10 megahertz to about 100 megahertz. In some examples, the power supply 224 may be configured to supply first electrical power having a first voltage to the electrolyte mixture 108 during a first period of time. In some examples, the power supply 224 may be configured to supply second electrical power having a second voltage to the electrolyte mixture 108 during a first period of time. In some examples, the power supply 224 may be configured to supply the first electrical power having a first current to the electrolyte mixture 108 during the first period of time. In some examples, the power supply 224 may be configured to supply the second electrical power having a second current to the electrolyte mixture 108 during the first period of time. In some examples, the power supply 224 may be configured to supply the first electrical power having a first frequency to the electrolyte mixture 108 during the first period of time. In some examples, the power supply 224 may be configured to supply the second electrical power having a second frequency to the electrolyte mixture 108 during the first period of time. In some examples, the second voltage may be different than the first voltage, the second current is different than the first current, and/or the second frequency is different than the first frequency. In some examples, the electrical power supplied by the power supply 224 may comprise a current that is between about 20 milliamperes to about 1000 milliamperes, such as between about 88 milliamperes to about 215 milliamperes. In some examples, the electrical power supplied by the power supply 224 may comprise a voltage that is between about 1 millivolt to about 100 millivolts, such as between about 8.16 millivolts to about 17.88 millivolts. In some examples, the electrical power supplied by the power supply 224 may comprise a frequency that is between about 1 megahertz to about 1000 megahertz, such as between about 10 megahertz to about 100 megahertz.

In some examples, the power supply 224 may comprise one or more displays (e.g., a first display 226, a second display 228, a third display 230, and/or a fourth display 232) to display one or more parameters. In some examples, the power supply 224 may be electrically connected to the first electrode 201 and/or the second electrode 202 via one or more connectors (e.g. a first connector 214, a second connector 216, etc.). In some examples, the first connector 214 may be metal connector (e.g., metal wire connector, metal cable connector, etc.) to provide the electrical power to the first electrode 201. In some examples, the second connector 216 may be metal connector (e.g., metal wire connector, metal cable connector, etc.) to provide the electrical power to the second electrode 202. In some examples, the first connector 214 may be non-metal conductive connector (e.g., graphite connector) to provide the electrical power to the first electrode 201. In some examples, the second connector 216 may be non-metal conductive connector (e.g., graphite connector) to provide the electrical power to the second electrode 202. The power supply 224 may comprise one or more outlets (e.g., a first outlet 209, a second outlet 207) wherein the one or more outlets may provide electrical power to the first electrode 201 via the first connector 214 and/or may provide electrical power to the second electrode 202 via the second connector 216. In some examples, the first outlet 209 may provide positive charge to the first electrode 201 and/or the second outlet 207 may provide negative charge to the second electrode 202. In some examples, the first outlet 209 may provide negative charge to the first electrode 201 and/or the second outlet 207 may provide positive charge to the second electrode 202. In some examples, the power supply 224 may comprise a power button 203 (e.g., power switch, On/Off button, etc.), wherein the power button 203 may turn on/off the power supply 224.

In some examples, the plasma production system 200 may comprise a controller 218. The controller 210 may comprise one or more manual input ports 220 (e.g., control knobs) and/or an automated input port 234 (e.g., universal serial bus (USB) ports). In some examples, the controller may be configured to apply the one or more parameters on the power supply 224. In some examples, the one or more parameters may comprise a first voltage, a first current, a first frequency, and/or a first period of time. In some examples, the one or more parameters may comprise a second voltage, a second current, a second frequency, and/or a second period of time.

FIG. 2B illustrates the plasma production system 200 at a first stage of operation, according to some embodiments. In some examples, the first stage of operation is when the power supply 224 supplies a first current in a first period of time, a first voltage in a first period of time and/or a first frequency in a first period of time. In some examples, the first electrode 201 may be a copper electrode and/or the second electrode 202 may comprise an electrode body 204 (e.g., a copper body) covered by the coating 206. In some examples, the first electrode 201 and/or the second electrode 202 may be at least partially surrounded by the electrolyte mixture 108. In some examples, the first electrode 201 may be an anode and/or the second electrode 202 may be a cathode. In some examples, the first electrode 201 may be a cathode and/or the second electrode 202 may be an anode. In some examples, the first electrode may be connected to the first outlet 209 via the first connector 214. The second electrode 202 may be connected to the second outlet 207 via the second connector 216. In some examples, the power supply 224 may apply a first electrical power to the first electrode 201 and/or the second electrode 202 in a first period of time. The first electrical power may be associated with the first voltage, the first current and/or the first frequency. In some examples, the first electrode 201 and/or the second electrode 202 may apply the first electrical power of the power supply 224 to the electrolyte mixture 108. The plasma production system 200 comprises a controller 218. The controller may determine the value of the first period of time, the value of the first voltage, the value of the first current and/or the value of the first frequency via the one or more manual input ports 220 and/or via the automated input port 234. The controller may send the value of the first period of time, the first voltage, the first current and/or the first frequency to the power supply 224. As shown in FIG. 2B, the value of the first current is shown in the first display 226 which is 215 milliamperes. The value of the first voltage is shown in the second display 228 which is 17.88 millivolts. The value of the first frequency is shown in the third display 230 which is 62 megahertz. The value of the first period of time is shown in the fourth display 232 which is 5 minutes. Other values of the first voltage, the first current, the first frequency and/or the first period of time are within the scope of the present disclosure.

In some examples, the first stage of operation may occur after switching on the power supply 224 via pushing the power button 203. In some examples, at the first stage of operation, a reduction reaction and/or an oxidation reaction may occur between the first electrode 201, the second electrode 202 and/or the electrolyte mixture 108. In some examples, the reduction reaction (e.g., composition reaction) may occur within the electrolyte mixture 108 adjacent to the second electrode 202. The reduction reaction may be as follows:

2H⁺+2e⁻→H₂

wherein H⁺ represents hydrogen cation (the cation of protium), e⁻ represents electron and H₂ represents hydrogen in form of gas. In some examples, the hydrogen may start to produce in the proximity of the second electrode 202 in form of a first plurality of hydrogen bubbles 236.

In some examples, the oxidation reaction (e.g., decomposition reaction) may occur within the electrolyte mixture 108 adjacent to the first electrode 201. The oxidation reaction may be as follows:

Cu→Cu²⁺+2e⁻

wherein Cu represents copper, e⁻ represents electron and Cu²⁺ represents copper cation.

In some examples, the first electrode 201 may be an anode and/or the second electrode 202 may be a cathode.

FIG. 2C illustrates the plasma production system 200 at a second stage of operation, according to some embodiments. In some examples, the second stage of operation may occur when the power supply 224 supplies the second current in the second period of time, the second voltage in the second period of time and/or the second frequency in the second period of time. In some examples, the first electrode 201 and/or the second electrode 202 may apply the second electrical power of the power supply 224 to the electrolyte mixture 108. The plasma production system 200 comprises a controller 218. The controller may determine the value of the second period of time, the value of the second voltage, the value of the second current and/or the value of the second frequency via the one or more manual input ports 220 and/or via the automated input port 234. The controller may send the value of the second period of time, the second voltage, the second current and/or the second frequency to the power supply 224. As shown in FIG. 2C, the value of the second current is shown in the first display 226 which is 88 milliamperes. The value of the second voltage is shown in the second display 228 which is 8.16 millivolts. The value of the second frequency is shown in the third display 230 which is 62 megahertz. The value of the second period of time is shown in the fourth display 232 which is 15000 minutes. Other values of the second voltage, the second current, the second frequency and/or the second period of time are within the scope of the present disclosure.

In some examples, the second stage of operation may occur after the second stage of operation. In some examples, at the second stage of operation, a reduction reaction and/or an oxidation reaction may occur between the first electrode 201, the second electrode 202 and/or the electrolyte mixture 108.

In some examples, the reduction reaction (e.g., composition reaction) may occur within the electrolyte mixture 108 adjacent to the second electrode 202. The reduction reaction may be as follows:

2H⁺+2e⁻→H₂

wherein H⁺ represents hydrogen cation (the cation of protium), e⁻ represents electron and H₂ represents hydrogen 242 in form of gas. In some examples, the produced hydrogen may exit the electrolyte mixture 108 in form of a second plurality of hydrogen bubbles 242. The second plurality of hydrogen bubbles 242 may exit the bioreactor tank 102 to a space outside the bioreactor tank 102.

In some examples, the oxidation reaction (e.g., decomposition reaction) may occur within the electrolyte mixture 108 adjacent to the first electrode 201. The oxidation reaction may be as follows:

Cu→Cu²⁺+2e⁻

wherein Cu represents copper, e⁻ represents electron and Cu²⁺ represents copper cation.

In an example, the first electrode 201 may be an anode and/or the second electrode 202 may be a cathode. In some examples, the first electrode 201 may comprise zinc (Zn) and/or the second electrode 202 may comprise the electrode body 204 and/or the coating 206. The electrode body 204 may comprise copper (Cu) and/or the coating may comprise copper oxide (CuO). In some examples, the reduction reaction (e.g., composition reaction) may occur within the electrolyte mixture 108 adjacent to the second electrode 202. The reduction reaction may be as follows:

Cu²⁺+2e⁻→Cu

wherein Cu represents copper, e⁻ represents electron and Cu²⁺ represents copper cation.

In some examples, the oxidation reaction (e.g., decomposition reaction) may occur within the electrolyte mixture 108 adjacent to the first electrode 201. The oxidation reaction may be as follows:

2H₂O→O₂+4H⁺+4e⁻

wherein H₂O represents water, e⁻ represents electron and H⁺ represents hydrogen cation (the cation of protium) and 02 represents oxygen in the form of gas.

In an example, the first electrode 201 may be an anode and/or the second electrode 202 may be a cathode. In some examples, the first electrode 201 may comprise iron (Fe) and/or the second electrode 202 may comprise the electrode body 204 and/or the coating 206. The electrode body 204 may comprise copper (Cu) and/or the coating may comprise copper oxide (CuO). In some examples, the reduction reaction (e.g., composition reaction) may occur within the electrolyte mixture 108 adjacent to the second electrode 202. The reduction reaction may be as follows:

Cu²⁺+2e⁻→Cu

wherein Cu represents copper, e⁻ represents electron and Cu²⁺ represents copper cation.

In some examples, the oxidation reaction (e.g., decomposition reaction) may occur within the electrolyte mixture 108 adjacent to the first electrode 201. The oxidation reaction may be as follows:

Fe→Fe²⁺+2e⁻

wherein Fe represents iron, e⁻ represents electron and Fe²⁺ represents iron cation.

In an example, the first electrode 201 may be a cathode and/or the second electrode 202 may be an anode. In some examples, the second electrode 202 may comprise gold (Au) and/or the first electrode 201 may comprise the electrode body 204 and/or the coating 206. The electrode body 204 may comprise copper (Cu) and/or the coating may comprise copper oxide (CuO). In some examples, the reduction reaction (e.g., composition reaction) may occur within the electrolyte mixture 108 adjacent to the first electrode 201. The reduction reaction may be as follows:

Au³⁺+3e⁻→Au

wherein Au represents gold, e⁻ represents electron and Au³⁺ represents gold cation.

In some examples, the oxidation reaction (e.g., decomposition reaction) may occur within the electrolyte mixture 108 adjacent to the second electrode 202. The oxidation reaction may be as follows:

Cu→Cu²⁺+2e⁻

wherein Cu represents copper, e⁻ represents electron and Cu²⁺ represents copper cation.

In an example, the first electrode 201 may be a cathode and/or the second electrode 202 may be an anode. In some examples, the second electrode 202 may comprise silver (Ag) and/or the first electrode 201 may comprise the electrode body 204 and/or the coating 206. The electrode body 204 may comprise copper (Cu) and/or the coating may comprise copper oxide (CuO). In some examples, the reduction reaction (e.g., composition reaction) may occur within the electrolyte mixture 108 adjacent to the first electrode 201. The reduction reaction may be as follows:

Ag⁺+e⁻→Ag

wherein Ag represents silver, e⁻ represents electron and Ag⁺ represents silver cation.

In some examples, the oxidation reaction (e.g., decomposition reaction) may occur within the electrolyte mixture 108 adjacent to the second electrode 202. The oxidation reaction may be as follows:

Cu→Cu²⁺+2e⁻

wherein Cu represents copper, e⁻ represents electron and Cu²⁺ represents copper cation.

In an example, the plasma production system 200 may comprise three electrodes. The three electrodes may comprise the first electrode 201, the second electrode 202 and/or an auxiliary electrode. In some examples, the first electrode 201 may be an anode, the second electrode 202 may be a cathode and/or the auxiliary electrode may be a cathode. In some examples the second electrode 202 may be connected to the auxiliary electrode via a connector. In some examples, the connector may be metal connector (e.g., metal wire connector, metal cable connector, etc.) to provide the electrical power to the auxiliary electrode. In some examples, the connector may be non-metal conductive connector (e.g., graphite connector) to provide the electrical power to the auxiliary electrode. In some examples, the first electrode 201 may comprise copper (Cu), the second electrode 202 may comprise the electrode body 204 and/or the coating 206. The electrode body 204 may comprise copper (Cu) and/or the coating may comprise copper oxide (CuO). In some examples, the auxiliary electrode may comprise zinc (Zn). In some examples, the reduction reaction (e.g., composition reaction) may occur within the electrolyte mixture 108 adjacent to the second electrode 202. The reduction reaction may be as follow:

Cu²⁺+2e⁻→Cu

wherein Cu represents copper, e⁻ represents electron and Cu²⁺ represents copper cation.

In some examples, the reduction reaction (e.g., composition reaction) may occur within the electrolyte mixture 108 adjacent to the auxiliary electrode. The reduction reaction may be as follow:

2H₂O+2e⁻→H₂+2OH⁻

wherein H₂O represents water, e⁻ represents electron and H₂ represents hydrogen and OH⁻ represents hydroxide.

In some examples, the oxidation reaction (e.g., decomposition reaction) may occur within the electrolyte mixture 108 adjacent to the first electrode 201. The oxidation reaction may be as follows:

Cu→Cu²⁺+2e⁻

wherein Cu represents copper, e⁻ represents electron and Cu²⁺ represents copper cation.

FIG. 2D illustrates the plasma production system 200 after the second stage of operation (e.g., when the power supply 224 is off), according to some embodiments. In some examples, after the second stage of operation, the power supply 224 may stop providing the second electrical power to the first electrode 201 and/or the second electrode 202. In some examples, at least a portion 254 of the first electrode 201 may have corrosion based upon an oxidation reaction. In some examples, at least a portion 256 of the second electrode 202 may have corrosion based upon a reduction reaction. As shown in FIG. 2D, a plasma activated liquid 252 (e.g., plasma activated water) may be produced from the electrolyte mixture 108. For example, performing the first stage of operation and/or the second stage of operation on the electrolyte mixture 108 produces the plasma activated liquid 252. In some examples, the plasma activated liquid 252 may comprise metal ions (e.g., copper ions), hydrogen ions H⁺, hydroxide OH⁻, mineral salt, distilled water. In some examples, the plasma activated liquid 252 may comprise positively charge ions (e.g., cations). In some examples, the plasma activated liquid 252 may comprise negatively charge ions (e.g., anions). In some examples, the plasma activated liquid 252 may comprise a solution (e.g., an aqueous solution) comprising electrons, protons, and/or various ions that are obtained at ambient temperature and/or atmospheric pressure.

FIG. 2E illustrates the plasma production system 200 after a third period of time, according to some embodiments. In some examples, the third period of time may be when the plasma production system 200 is left to evaporate the plasma activated liquid 252. In some examples, the third period of time may occur after the second stage of operation when the power supply is off. As shown in FIG. 2E, liquid of the plasma activated liquid 252 (shown in FIG. 2D) evaporates to produce a plasma activated sediment 262 (e.g., plasma activated dust). In some examples, the plasma activated sediment 262 may comprise metal ions (e.g., copper ions), hydrogen ions H⁺, hydroxide OH⁻, mineral salt. In some examples, the plasma activated sediment 262 may comprise positively charge ions (e.g., cations). In some examples, the plasma activated sediment 262 may comprise negatively charge ions (e.g., anions).

FIG. 2F illustrates a real image of the plasma activated liquid 252 (shown in FIG. 2D) produced by the plasma production system 200, according to some embodiments. In some examples, the plasma activated liquid 252 may be a plasma activated product. In some examples, the plasma activated liquid 252 may be a plasma activated medicine. In some examples, the plasma activated liquid 252 may be produced by using the plasma production system 200, such as using one or more techniques provided herein with respect to the plasma production system 200 of FIGS. 2A-2E. In some examples, the plasma activated liquid 252 may be diluted via using a liquid in order to obtain a right dosage of a medicine to be administered to a specific patient. In an example, the liquid may comprise distilled water. In some examples, the plasma activated liquid 252 may be used to treat a patient, wherein the plasma activated liquid 252 may be administered to the patient via one or more treatment types. The one or more treatment types may comprise administration via injection, administration via inhalation, and/or administration via skin absorption. In some examples, the administration via injection may comprise intravenous (IV) injection and/or subcutaneous (SC) injection (e.g., injection near a tumor, injection inside a tumor). In some examples, the administration via inhalation may be performed by utilizing an inhaler device (e.g., nebulizer). In some examples, the administration via skin absorption may be performed by combining the plasma activated liquid 252 with an ointment. In some examples, the administration via skin absorption may be performed by using a tub (e.g., filled with water) in which the plasma activated liquid 252 is poured inside the tub. In some examples, the administration via skin absorption may be performed via using a Balneotherapy technique. In some examples, the plasma activated liquid 252 may be used to treat one or more diseases corresponding to one or more patients. In some examples, the one or more diseases may comprise all cancer categories (e.g., carcinoma, sarcoma, leukemia, lymphoma, myeloma, brain and/or spinal cord cancers, etc.), diabetes (e.g., type 1 and/or type 2 diabetes), elevated liver enzymes, infectious wounds, genital wards (e.g., diseases caused by Human Papillomavirus (HPV)), oral condition (e.g., diseases, dental caries, periodontal diseases, etc.), autoimmune diseases (e.g., Scleroderma, etc.), foot ulcers (e.g., diabetic foot ulcers), pressure ulcers (e.g., pressure sores, bed sores, etc.), infectious diseases (e.g., Covid-19, chicken pox, etc.), skin diseases (e.g., acne, Eczema, etc.), Epidermolysis Bullosa (EB) disease, sinusitis diseases (e.g., chronic sinusitis), ear infection diseases (e.g., chronic inner ear infections, chronic middle ear infections), sinusitis (e.g., chronic sinusitis), eye infection diseases, bone infection diseases. In some examples, the plasma activated liquid 252 may be used in palliative care corresponding to one or more patients. The plasma activated liquid 252 may be used as an analgesics medicine (e.g., a painkiller medicine). In some examples, the plasma activated liquid 252 may be used as a medicine to treat bacteria, viruses, fungi and/or parasites which are no longer respond to antimicrobial medicines. In some examples, the plasma activated liquid 252 may be used to treat all types of bacteria and/or fungi.

In some examples, the plasma activated liquid 252 may be used as a non-alcoholic disinfectant. In some examples, the plasma activated liquid 252 may be used as a sterilizer (e.g., for surfaces, medical equipment, etc.). In some examples, the plasma activated liquid 252 may be used as an agricultural reinforcement fertilizer. In some examples, the plasma activated liquid 252 may be used as a biocompatible pesticide.

FIG. 2G illustrates a real image of the plasma activated sediment 262 (shown in FIG. 2E) produced by the plasma production system 200, according to some embodiments. In some examples, the plasma activated sediment 262 may be mixed with a liquid (e.g. distilled water). In some examples, mixing the plasma activated sediment 262 with the liquid produces a plasma activated liquid (e.g., the plasma activated liquid 252). In some examples, the plasma activated sediment 262 may be a powdered plasma activated sediment (e.g., comprising a powder). In some examples, the powdered plasma activated sediment may be used to treat one or more patients, wherein the powdered plasma activated sediment may be administered to the one or more patients via one or more treatment types. The one or more treatment types may comprise administration via inhalation, and/or administration via skin absorption. In some examples, the administration via inhalation may be performed by utilizing an inhaler device (e.g., nebulizer). In some examples, the administration via skin absorption may be performed by combining the powdered plasma activated sediment with an ointment. In some examples, the administration via skin absorption may be performed by using a tub (e.g., filled with water) in which the powdered plasma activated sediment is poured inside the tub. In some examples, the administration via skin absorption may be performed via using a Balneotherapy technique.

Thus, in accordance with some embodiments, the plasma production system 200 may be used to produce the plasma activated product comprising at least one of the plasma activated liquid 252 (e.g., plasma activated water), the plasma activated sediment 262, powdered plasma activated sediment, a plasma, a cold atmospheric plasma, a biocompatible plasma, etc.

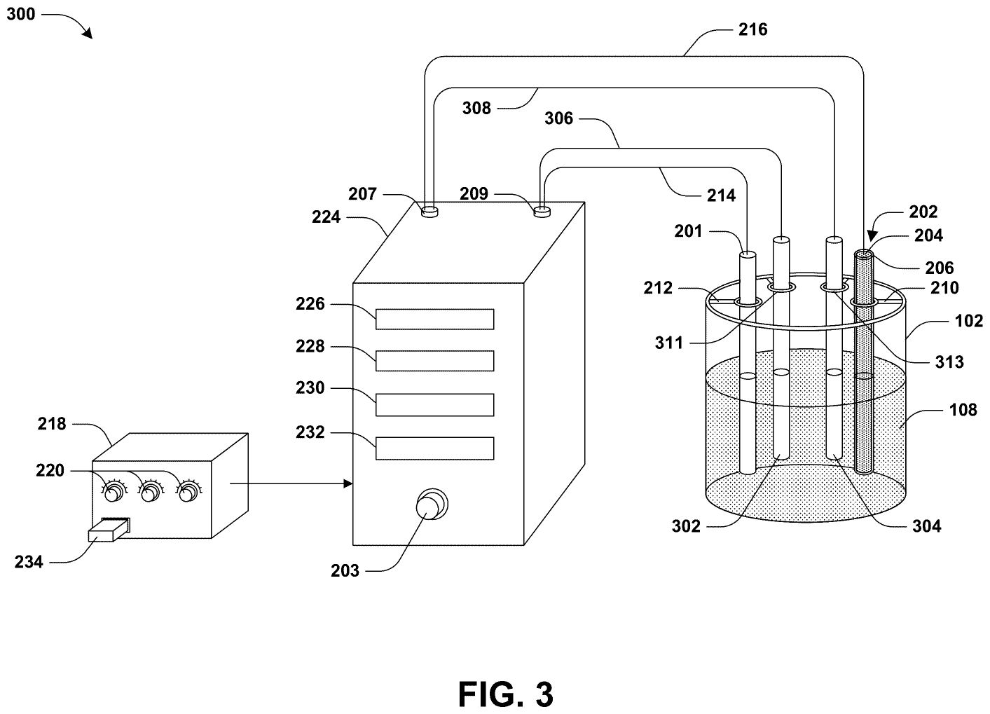

FIG. 3 illustrates an example plasma production system 300 utilizing a plurality of electrodes, according to some embodiments. In an example, a plasma activated product (e.g., at least one of a plasma activated liquid, a plasma activated sediment, a plasma, a cold atmospheric plasma, a biocompatible plasma, etc.) is produced using the example plasma production system 300, such as using one or more techniques provided herein with respect to the plasma production system 200 of FIGS. 2A-2E. The example plasma production system 300 comprises one or more first electrodes (e.g., the first electrode 201, a third electrode 302). The one or more first electrodes may comprise one or more metals (e.g., copper (Cu), gold (Au), silver (Ag), zinc (Zn), iron (Fe) and/or one or more non-metals (e.g., graphite (Gr)). The example plasma production system 300 comprises one or more second electrodes (e.g., the second electrode 202, a fourth electrode 304). In some examples, the third electrode 302 may be connected to the first outlet 209 via the third connector 306 and/or the fourth electrode 304 may be connected to the second outlet 207 via the fourth connector 308. In some examples, the third electrode 302 may be connected to the first electrode 201 via a third connector 306 and/or the fourth electrode 304 may be connected to the second electrode 202 via a fourth connector 308. In some examples, the third electrode 302 may be connected to the first connector 214 via the third connector 306 and/or the fourth electrode 304 may be connected to the second connector 216 via the fourth connector 308. In some examples, the third electrode 302 and/or the fourth electrode 304 may be at least partially surrounded by the electrolyte mixture 108. In some examples, the third electrode 302 may be held by a third holder 311 and/or the fourth electrode 304 may be held by a fourth holder 313. In some examples, the third holder 311 and/or the fourth holder 313 may be attached to the bioreactor tank 102. In some examples, the one or more first electrodes and/or the one or more second electrodes may comprise one or more metals (e.g., copper (Cu), gold (Au), silver (Ag), zinc (Zn), iron (Fe) and/or one or more non-metals (e.g., graphite (Gr)). In some examples, one or more electrodes of the one or more first electrodes may be covered by a coating (e.g., the coating 206). In some examples, one or more electrodes of the one or more second electrodes may be covered by a coating (e.g., the coating 206).

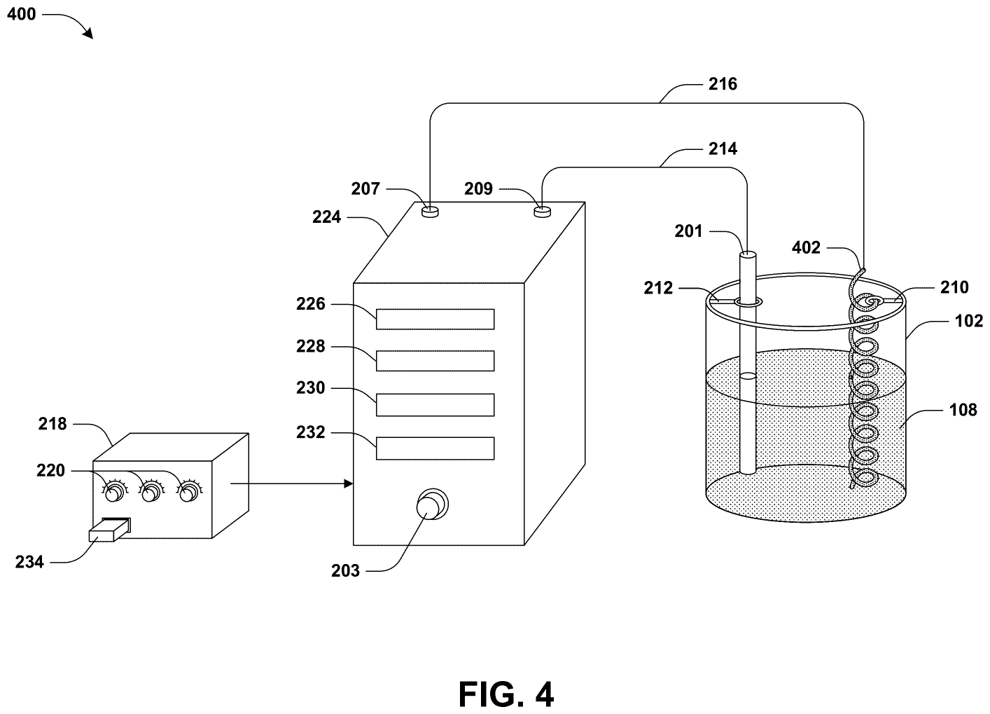

FIG. 4 illustrates an example plasma production system 400 utilizing an electrode as an anode and/or a coil-shaped electrode 402 as a cathode, according to some embodiments. In an example, a plasma activated product (e.g., at least one of a plasma activated liquid, a plasma activated sediment, a plasma, a cold atmospheric plasma, a biocompatible plasma, etc.) is provided. The plasma activated product may be produced by using the example plasma production system 400, such as using one or more techniques provided herein with respect to the plasma production system 200 of FIGS. 2A-2E. The example plasma production system 400 comprises the first electrode 201 and/or the coil-shaped electrode 402. In some examples, the coil-shaped electrode 402 may be connected to the second outlet 209 via the second connector 216. In some examples, the coil-shaped electrode 402 may be at least partially surrounded by the electrolyte mixture 108. In some examples, the coil-shaped electrode 402 may be held by a second holder 210. In some examples, the second holder 210 may be attached to the bioreactor tank 102. In some examples, the coil-shaped electrode 402 may comprise one or more metals (e.g., copper (Cu), gold (Au), silver (Ag), zinc (Zn), iron (Fe) and/or one or more non-metals (e.g., graphite (Gr)). In some examples, the coil-shaped electrode 402 may be covered by a coating (e.g., the coating 206 shown in FIG. 2A-2E).

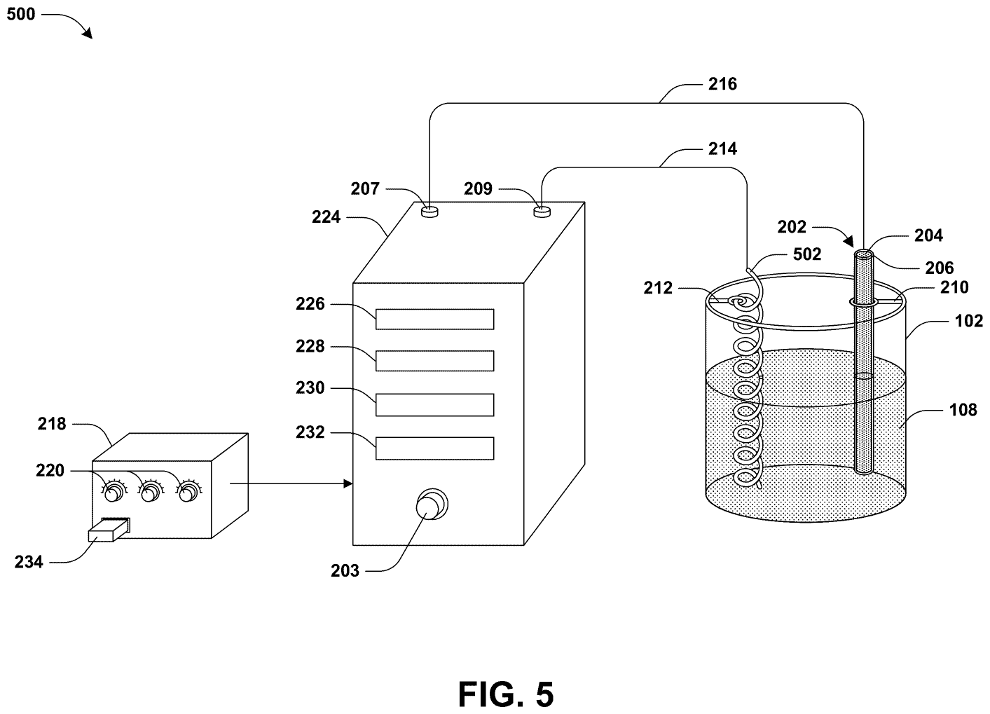

FIG. 5 illustrates an example plasma production system 500 utilizing an electrode as a cathode and/or a coil-shaped electrode 502 as an anode, according to some embodiments. In an example, a plasma activated product (e.g., at least one of a plasma activated liquid, a plasma activated sediment, a plasma, a cold atmospheric plasma, a biocompatible plasma, etc.) is provided. The plasma activated product may be produced by using the example plasma production system 500, such as using one or more techniques provided herein with respect to the plasma production system 200 of FIGS. 2A-2E. The example plasma production system 500 comprises the second electrode 202 and/or the coil-shaped electrode 502. In some examples, the coil-shaped electrode 502 may be connected to the first outlet 209 via the first connector 214. In some examples, the coil-shaped electrode 502 may be at least partially surrounded by the electrolyte mixture 108. In some examples, the coil-shaped electrode 502 may be held by a first holder 212. In some examples, the first holder 212 may be attached to the bioreactor tank 102. In some examples, the coil-shaped electrode 502 may comprise one or more metals (e.g., copper (Cu), gold (Au), silver (Ag), zinc (Zn), iron (Fe) and/or one or more non-metals (e.g., graphite (Gr)). In some examples, the coil-shaped electrode 502 may be covered by a coating (e.g., the coating 206 shown in FIG. 2A-2E).

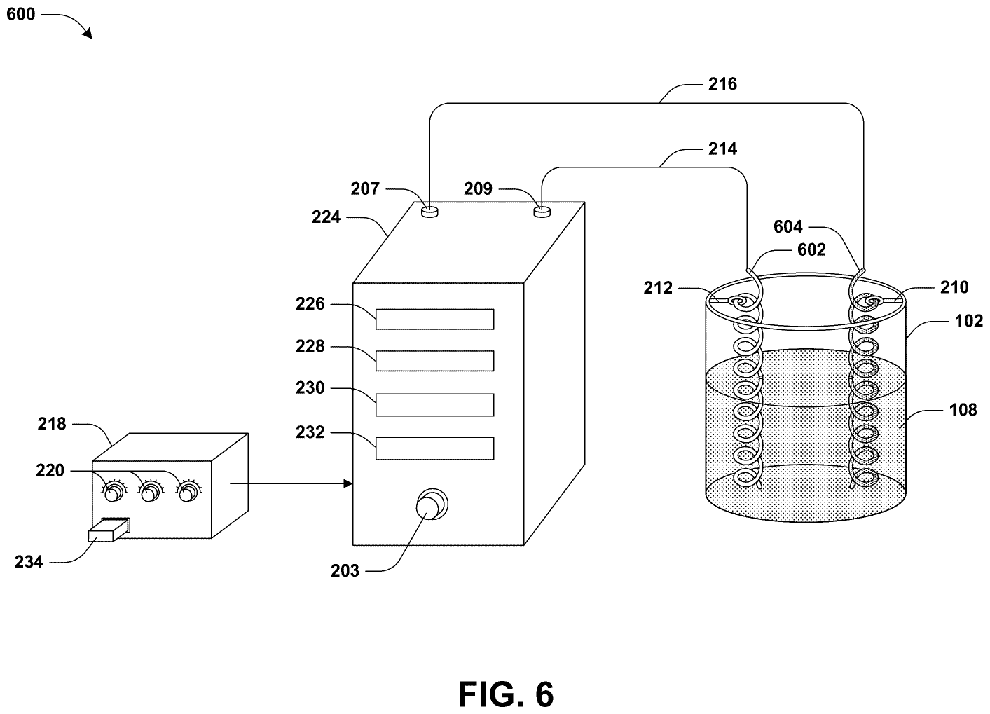

FIG. 6 illustrates an example plasma production system 600 utilizing a first coil-shaped electrode 602 as an anode and/or a second coil-shaped electrode 604 as an anode, according to some embodiments. In an example, a plasma activated product (e.g., at least one of a plasma activated liquid, a plasma activated sediment, a plasma, a cold atmospheric plasma, a biocompatible plasma, etc.) is provided. The plasma activated product may be produced by using the example plasma production system 600, such as using one or more techniques provided herein with respect to the plasma production system 200 of FIGS. 2A-2E. The example plasma production system 600 comprises the first coil-shaped electrode 602 and/or the second coil-shaped electrode 604. In some examples, the first coil-shaped electrode 602 may be connected to the first outlet 209 via the first connector 214. In some examples, the second coil-shaped electrode 604 may be connected to the second outlet 207 via the second connector 216. In some examples, the first coil-shaped electrode 602 may be at least partially surrounded by the electrolyte mixture 108. In some examples, the second coil-shaped electrode 604 may be at least partially surrounded by the electrolyte mixture 108. In some examples, the first coil-shaped electrode 602 may be held by the first holder 212. In some examples, the second coil-shaped electrode 604 may be held by the second holder 210. In some examples, the first holder 212 and/or the second holder 212 may be attached to the bioreactor tank 102. In some examples, the first coil-shaped electrode 602 and/or the second coil-shaped electrode 604 may comprise one or more metals (e.g., copper (Cu), gold (Au), silver (Ag), zinc (Zn), iron (Fe) and/or one or more non-metals (e.g., graphite (Gr)). In some examples, the first coil-shaped electrode 602 may be covered by a coating (e.g., the coating 206 shown in FIG. 2A-2E). In some examples, the second coil-shaped electrode 604 may be covered by a coating (e.g., the coating 206 shown in FIG. 2A-2E).

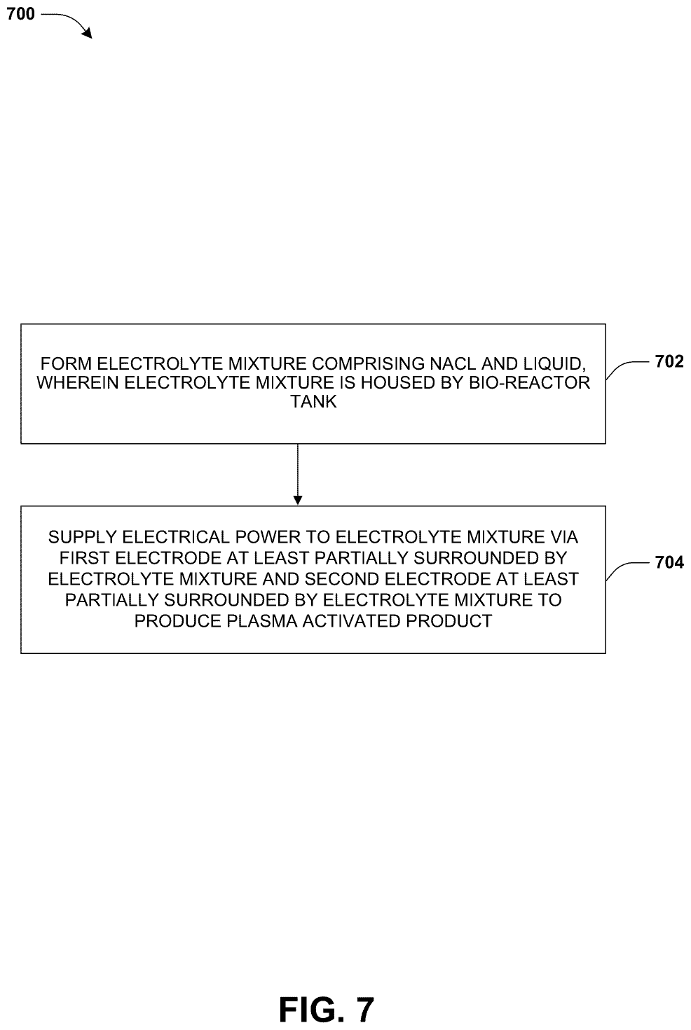

A method 700 of producing a plasma activated product (e.g., at least one of a plasma activated liquid, a plasma activated sediment, a plasma, a cold atmospheric plasma, a biocompatible plasma, etc.), is illustrated in FIG. 7 in accordance with some embodiments. At 702, an electrolyte mixture (e.g., the electrolyte mixture 108) is formed, wherein the electrolyte mixture comprises sodium chloride (NaCl) and/or a liquid. The electrolyte mixture is housed by a bioreactor tank (e.g., the bioreactor tank 102). At 704, an electrical power is supplied to the electrolyte mixture via a first electrode (e.g., the first electrode 201) and/or via a second electrode (e.g., the second electrode 202) to produce the plasma activated product. In some examples, the first electrode is at least partially surrounded by the electrolyte mixture. The second electrode is at least partially surrounded by the electrolyte mixture.

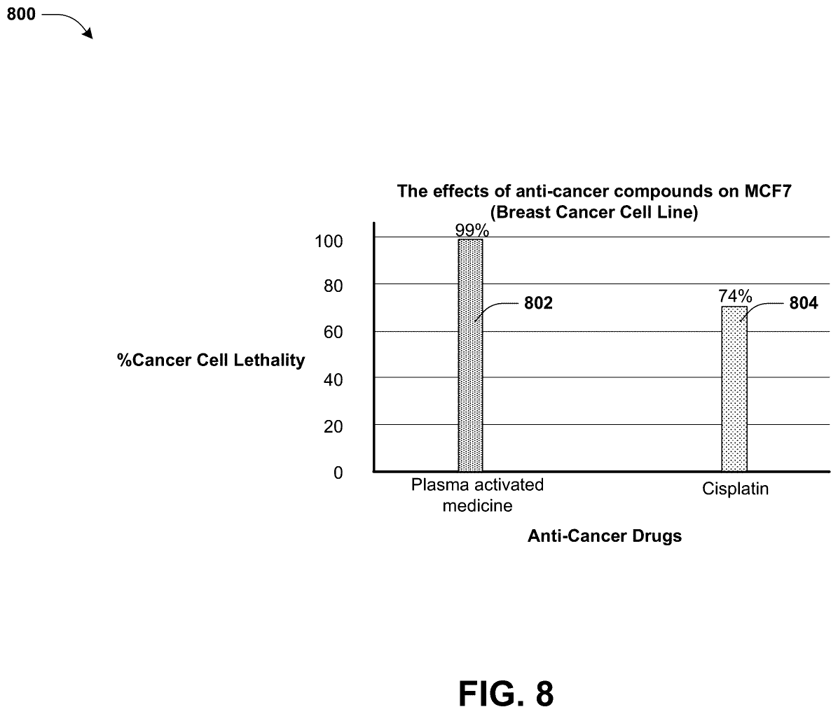

FIG. 8 presents a chart 800 displaying cancer cell lethality on Breast Cancer Cell Line (MCF7) corresponding to anti-cancer drugs, according to some embodiments. The chart 800 comprises a first cancer cell lethality percentage 802 representative of cancer cell lethality corresponding to a plasma activated product (e.g., at least one of a plasma activated liquid, a plasma activated sediment, a plasma, a cold atmospheric plasma, a biocompatible plasma, etc.) and/or a second cancer cell lethality percentage 804 representative of cancer cell lethality corresponding to Cisplatin. In some examples, the plasma activated product exhibited in the chart 800 is produced using one or more of the techniques provided herein, such as using the plasma production system 200 in accordance with one or more of the techniques shown in and/or described with respect to FIGS. 2A-2G, using example plasma production systems in accordance with one or more of the techniques shown in and/or described with respect to FIGS. 3-6, and/or using the method 700 in accordance with one or more of the techniques shown in and/or described with respect to FIG. 7. In some examples, the plasma activated product may be used as a medicine (e.g., a plasma activated medicine). The chart 800 is based upon testing the plasma activated product and Cisplatin on cancer cells (e.g., Breast Cancer Cell Line (MCF7)). The chart 800 illustrates that the first cancer cell lethality percentage 802 representative of cancer cell lethality corresponding to the plasma activated product is about 99%. The chart 800 illustrates that the second cancer cell lethality percentage 804 representative of cancer cell lethality corresponding to Cisplatin is about 74%. The chart 800 shows that the effectiveness of plasma activated product in killing the cancer cells (e.g., Breast Cancer Cell Line (MCF7)) is about 25% more than the effectiveness of Cisplatin in killing the cancer cells (e.g., Breast Cancer Cell Line (MCF7)). In some examples, the plasma activated product kills less normal and/or healthy human cells than Cisplatin.

In some examples, in order for the plasma activated product to enter a defected cell (e.g., a cancer cell of a human), the plasma activated product may first interact with a cell membrane and then may enter the defected cell. The plasma activated product having electromagnetic fields with the appropriate frequency and intensity may be used to enter the defected cell. The electromagnetic fields may stimulate the defected cell to open the combined permeable channels and/or insert receptors of the type of membrane receptors. As a result, when the plasma activated product enters the defected cell, its effect on the defected cell may be increased and the defected cell function may be better.

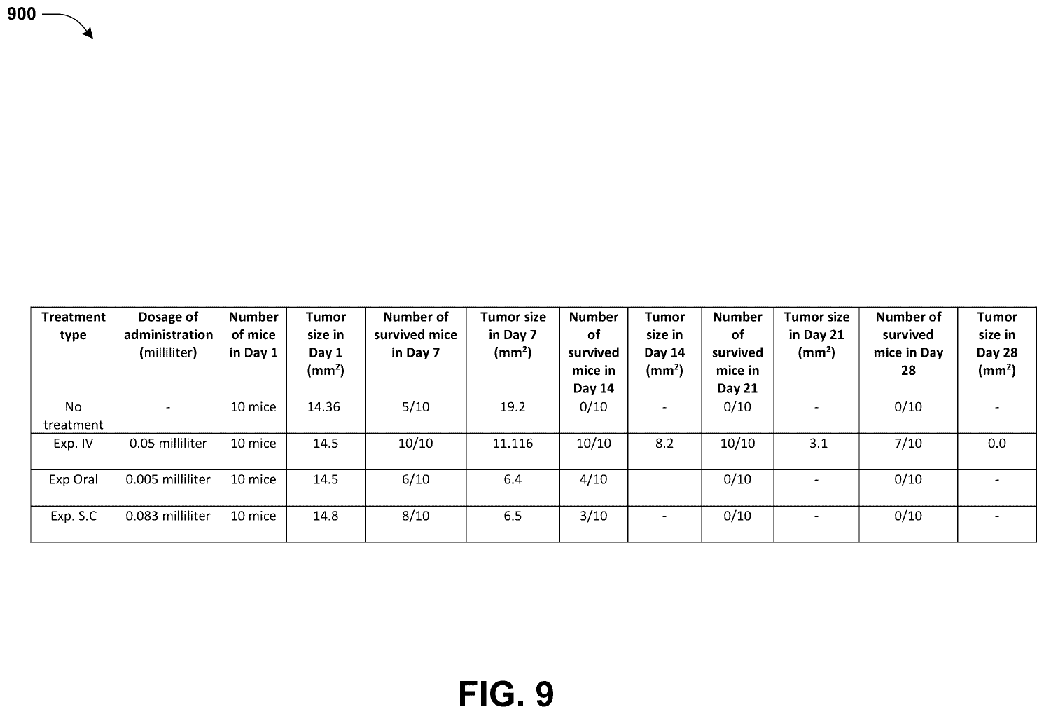

FIG. 9 presents a table 900 of animal testing parameters associated with treatment effectiveness of a plasma activated product (e.g., at least one of a plasma activated liquid, a plasma activated sediment, a plasma, a cold atmospheric plasma, a biocompatible plasma, etc.) corresponding to different types of administration, according to some embodiments. In some examples, the plasma activated product exhibited in the table 900 is produced using one or more of the techniques provided herein, such as using the plasma production system 200 in accordance with one or more of the techniques shown in and/or described with respect to FIGS. 2A-2G, using example plasma production systems in accordance with one or more of the techniques shown in and/or described with respect to FIGS. 3-6, and/or using the method 700 in accordance with one or more of the techniques shown in and/or described with respect to FIG. 7. In some examples, the plasma activated product may be used as a medicine (e.g., a plasma activated medicine). The animal testing parameters comprise treatment type, dosage of administration, number of mice (e.g., Wistar rats) in day 1, tumor size (e.g. cross sectional area of tumor) in day 1, number of survived mice in day 7, tumor size in day 7, number of survived mice in day 14, tumor size in day 14, number of survived mice in day 21, tumor size in day 21, number of survived mice in day 28, tumor size in day 28. The treatment type comprises intravenous administration (Exp. IV), oral administration (Exp. Oral), subcutaneous administration (Exp. S.C) and with no treatment. The dosage of administration for Exp. IV is 0.05 milliliter per 48-hours, the dosage of administration for Exp. Oral is 0.005 milliliter per 48-hours, the dosage of administration for Exp. S.C is 0.083 milliliter per 48-hours and there is no administration dosage for no treatment. There are 10 mice for each type of treatment. Tumor size in day 1 for each mouse of 10 mice corresponding to no treatment group is about 14.36 millimeter square (mm²). Tumor size in day 1 for each mouse of 10 mice corresponding to group Exp. IV is about 14.5 millimeter square (mm²). Tumor size in day 1 for each mouse of 10 mice corresponding to group Exp. Oral is about 14.5 millimeter square (mm²). Tumor size in day 1 for each mouse of 10 mice corresponding to group Exp. S.C is about 14.8 millimeter square (mm²). The table 900 shows that in day 7, there are 5 alive mice out of 10 corresponding to no treatment group, there are 10 alive mice out of 10 corresponding to group Exp. IV, there are 6 alive mice out of 10 corresponding to group Exp. Oral and there are 8 alive mice out of 10 corresponding to group Exp. S.C. Tumor size in day 7 for each alive mouse corresponding to no treatment group is about 19.2 millimeter square (mm²). Tumor size in day 7 for each alive mouse corresponding to group Exp. IV is about 11.116 millimeter square (mm²). Tumor size in day 7 for each alive mouse corresponding to group Exp. Oral is about 6.4 millimeter square (mm²). Tumor size in day 7 for each alive mouse corresponding to group Exp. S.C is about 6.5 millimeter square (mm²). The table 900 shows that in day 14, there is no alive mice corresponding to no treatment group, group Exp. Oral and group Exp. S.C. There are 10 alive mice out of 10 corresponding to group Exp. IV. Tumor size in day 14 for each alive mouse corresponding to group Exp. IV is about 8.2 millimeter square (mm²). The table 900 shows that in day 21, there are 10 alive mice out of 10 corresponding to group Exp. IV. Tumor size in day 21 for each alive mouse corresponding to group Exp. IV is about 3.1 millimeter square (mm²). The table 900 shows that in day 28, there are 7 alive mice out of 10 corresponding to group Exp. IV. Tumor size in day 28 for each alive mouse corresponding to group Exp. IV is about zero which indicates the remaining mice have no tumors and are cured.

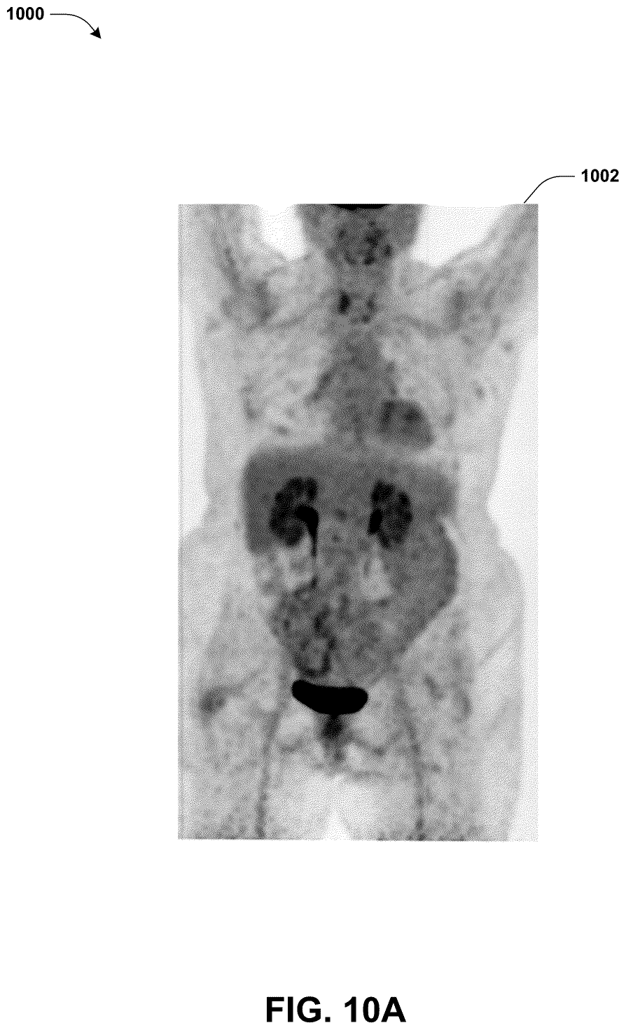

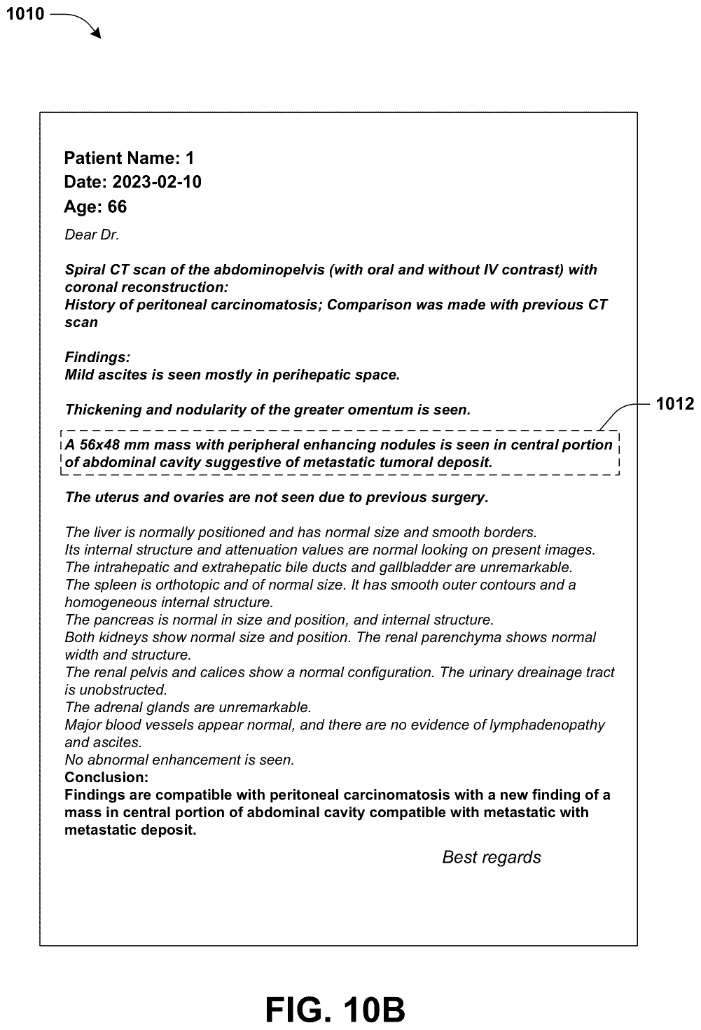

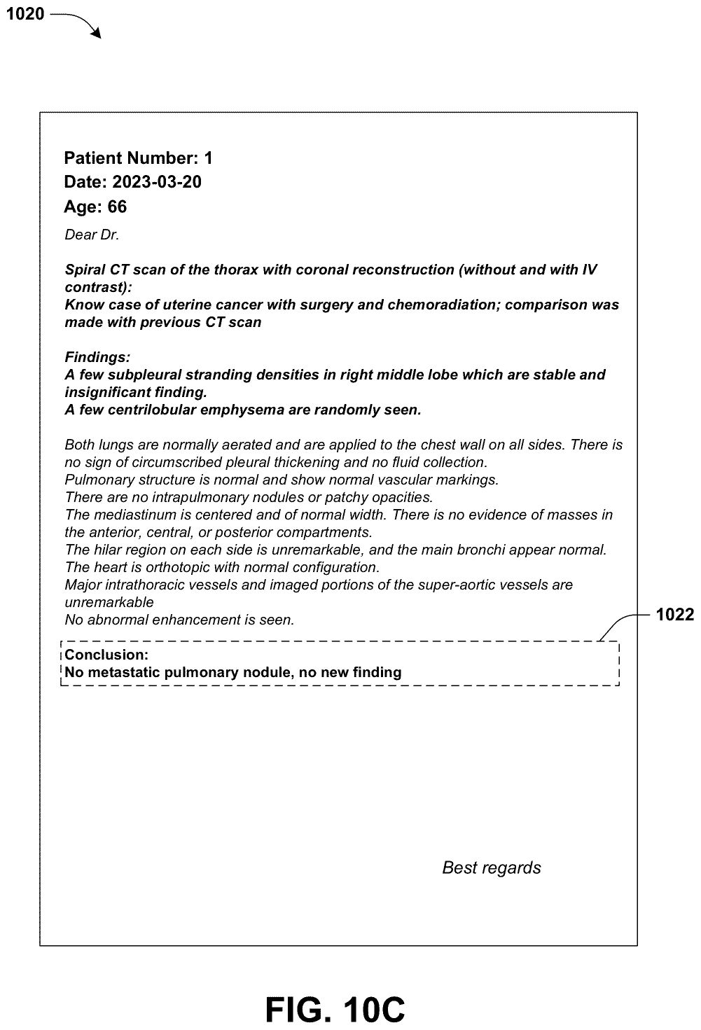

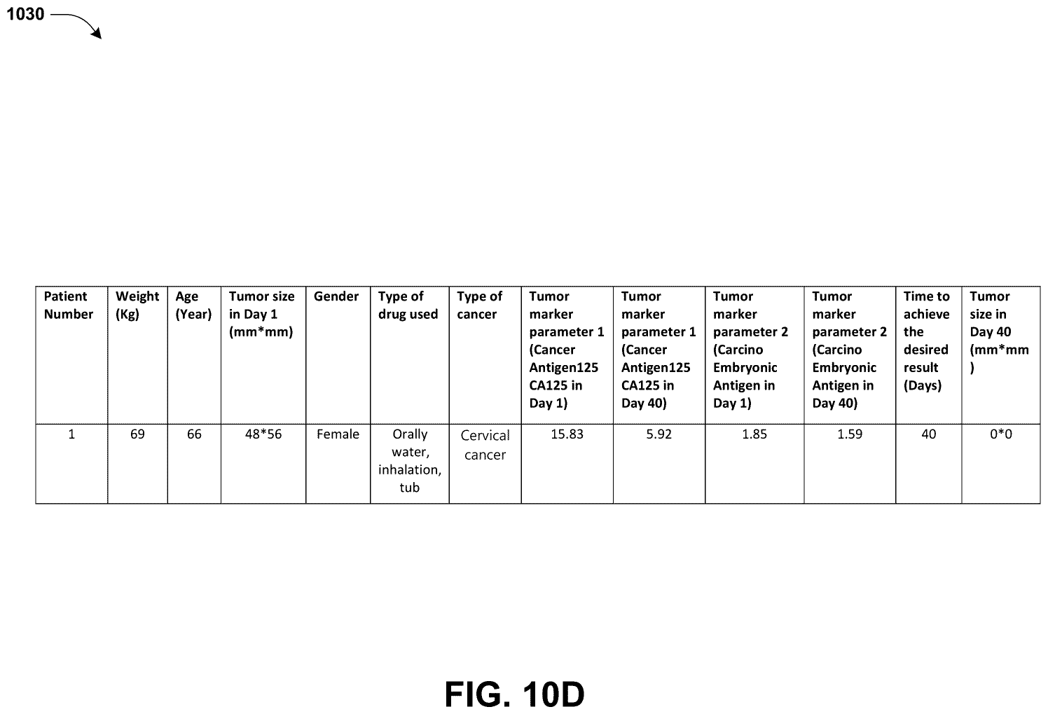

FIGS. 10A-10D illustrate an example scenario 1000 for treating a cancer patient via utilizing a plasma activated product (e.g., at least one of a plasma activated liquid, a plasma activated sediment, a plasma, a cold atmospheric plasma, a biocompatible plasma, etc.). In some examples, the plasma activated product exhibited in the example scenario 1000 is produced using one or more of the techniques provided herein, such as using the plasma production system 200 in accordance with one or more of the techniques shown in and/or described with respect to FIGS. 2A-2G, using example plasma production systems in accordance with one or more of the techniques shown in and/or described with respect to FIGS. 3-6, and/or using the method 700 in accordance with one or more of the techniques shown in and/or described with respect to FIG. 7. In some examples, the plasma activated product may be used as a medicine (e.g., a plasma activated medicine).

FIG. 10A presents an image 1002 of positron emission tomography (PET) scan corresponding to the cancer patient, according to some embodiments. The image 1002 shows the advanced stage of cancer cells growth (e.g. metastasis) corresponding to the cancer patient.

FIG. 10B presents a first report 1010 of a first computed tomography (CT) scan corresponding to a cancer patient before administering the plasma activated product to the cancer patient, according to some embodiments. The first report 1010 comprises a section 1012 wherein the section 1012 indicates “A 56×48 millimeter square (mm²) mass with peripheral enhancing nodules is seen in central portion of abdominal cavity suggestive of metastatic tumoral deposit.”.

FIG. 10C presents a second report 1020 of a second computed tomography (CT) scan corresponding to a cancer patient after administering the plasma activated product to the cancer patient, according to some embodiments. The second report 1020 comprises a section 1022 wherein the section 1022 indicates “No metastatic pulmonary nodule, no new finding”.

FIG. 10D presents a table 1030 of human testing report associated with tumor marker parameters before and after administering the plasma activated product corresponding to the cancer patient, according to some embodiments. The human testing report comprises weight, Age, gender, tumor size in day 1, type of administration, type of cancer, tumor marker parameter 1 in day 1, tumor marker parameter 2 in day 1, tumor marker parameter 1 in day 40, tumor marker parameter 2 in day 40, time to achieve the desired result, and tumor size in day 40, corresponding to the cancer patient. The type of cancer corresponding to the cancer patient is cervical cancer. The gender of the cancer patient is female. The time to achieve the desired result is 40 days. The weight of the cancer patient is 69 Kilograms. The age of the cancer patient is 66 years. The treatment type shown in the table 1030 is orally. The amount of the plasma activated product for each-day administering is 3 milliliters which is diluted by 1.5 liters of distilled water. The diluted plasma activated product is administered to the cancer patient orally. The dosage of administration to the cancer patient is 330 milliliters per 4-hours. In some examples, type of administration may change according to type of cancer and stages of cancer advancement. Other types of administration are within the scope of the present disclosure. The tumor marker parameter 1 in the table 1030 is cancer antigen 125 CA 125 and the tumor marker parameter 2 is Carcino Embryonic Antigen. The tumor marker parameter 1 in day 1 is about 15.83 and the tumor marker parameter 2 in day 1 is about 1.85. The tumor marker parameter 1 in day 40 is about 5.92 and the tumor marker parameter 2 in day 40 is about 1.59. The tumor size (e.g., cross sectional area of a tumor) in day 1 is about 48*56 millimeter square (mm²). The tumor size in day 40 is about zero which indicates the cancer patient has no tumors and is cured.

According to some embodiments, a plasma production system is provided. The plasma production system includes a bioreactor tank housing: an electrolyte mixture including sodium chloride (NaCl) and a liquid; a first electrode at least partially surrounded by the electrolyte mixture; and a second electrode at least partially surrounded by the electrolyte mixture. The plasma production system includes a power supply electrically connected to the first electrode and the second electrode. The power supply is configured to supply electrical power to the electrolyte mixture via the first electrode and the second electrode to produce a plasma activated product.

According to some embodiments, the power supply includes at least one of: a Direct Current (DC) power supply configured to supply DC electrical power to the electrolyte mixture; or an Alternating Current (AC) power supply configured to supply AC electrical power to the electrolyte mixture.

According to some embodiments, the power supply is configured to: supply first electrical power having a first voltage to the electrolyte mixture during a first period of time; and supply second electrical power having a second voltage to the electrolyte mixture during a second period of time, wherein the second voltage is different than the first voltage.

According to some embodiments, the power supply is configured to: supply first electrical power having a first current to the electrolyte mixture during a first period of time; and supply second electrical power having a second current to the electrolyte mixture during a second period of time, wherein the second current is different than the first current.

According to some embodiments, the power supply is configured to: supply first electrical power having a first frequency to the electrolyte mixture during a first period of time; and supply second electrical power having a second frequency to the electrolyte mixture during a second period of time, wherein the second frequency is different than the first frequency.

According to some embodiments. The power supply is configured to: supply first electrical power having a first voltage, a first current and a first frequency to the electrolyte mixture during a first period of time; and supply second electrical power having a second voltage, a second current and a second frequency to the electrolyte mixture during a second period of time, wherein at least two of: the second voltage is different than the first voltage; the second current is different than the first current; or the second frequency is different than the first frequency.

According to some embodiments, at least one of: a ratio of the sodium chloride to the liquid by weight is between about 0.01:1 to about 0.2:1; or a ratio of the sodium chloride to the liquid by weight is between about 0.05:1 to about 0.1:1.

According to some embodiments, at least one of: the liquid includes distilled water; or the plasma activated product includes at least one of a cold atmospheric plasma or a biocompatible plasma.

According to some embodiments, at least one of: the electrical power supplied by the power supply has a current that is at least one of: between about 20 milliamperes to about 1000 milliamperes; or between about 88 milliamperes to about 215 milliamperes; the electrical power supplied by the power supply has a voltage that is at least one of: between about 1 millivolt to about 100 millivolts; or between about 8.16 millivolts to about 17.88 millivolts; or the electrical power supplied by the power supply has a frequency that is at least one of: between about 1 megahertz to about 1000 megahertz; or between about 10 megahertz to about 100 megahertz.

According to some embodiments, the first electrode includes at least one of: copper (Cu); gold (Au); silver (Ag); zinc (Zn); iron (Fe); or graphite (Gr).

According to some embodiments, the second electrode includes: an electrode body; and a coating covering at least a portion of the electrode body, wherein the coating is at least partially surrounded by the electrolyte mixture.

According to some embodiments, the coating includes at least one of: a metal oxide layer; or a graphite layer.

According to some embodiments, the electrode body includes at least one of a metal or a graphite (Gr).

According to some embodiments, the metal includes at least one of: copper (Cu); gold (Au); silver (Ag); zinc (Zn); or iron (Fe).

According to some embodiments, the second electrode includes: a copper (Cu) body; or a copper oxide coating covering at least a portion of the copper body, wherein the copper oxide coating is at least partially surrounded by the electrolyte mixture.

According to some embodiments, the plasma production system includes at least one of: one or more first electrodes to which the first electrode is connected, wherein the one or more first electrodes include at least one of: copper (Cu); gold (Au); silver (Ag); zinc (Zn); iron (Fe); or graphite (Gr); or one or more second electrodes to which the second electrode is connected, wherein the one or more second electrodes include at least one of: copper (Cu); gold (Au); silver (Ag); zinc (Zn); iron (Fe); or graphite (Gr).

According to some embodiments, a method of treating a patient is provided. The method includes treating the patient using the plasma production product, wherein the treating the patient includes administering the plasma production product to the patient at least one of via injection, via inhalation, via oral administration or via skin absorption.

According to some embodiments, treating the patient includes at least one of: treating cancer using the plasma production product; treating diabetes using the plasma production product; regulating liver enzymes using the plasma production product; treating an infectious wound using the plasma production product; treating a genital wart using the plasma production product; treating an oral condition using the plasma production product; treating Scleroderma using the plasma production product; treating a foot ulcer using the plasma production product; treating a bed sore using the plasma production product; treating an infectious disease using the plasma production product; relieving pain using the plasma production product; treating an antibiotic-resistant virus using the plasma production product; treating bacteria using the plasma production product; treating a fungus using the plasma production product; treating a skin disease using the plasma production product; treating Epidermolysis Bullosa (EB) using the plasma production product; treating sinusitis using the plasma activated product; treating ear infection using the plasma activated product; treating an eye infection using the plasma production product; or treating a bone infection using the plasma production product.

According to some embodiments, a method is provided. The method includes applying a non-alcoholic disinfectant including the plasma production product; applying a sterilizer including the plasma production product; applying an agricultural reinforcement fertilizer including the plasma production product; or applying a pesticide including the plasma production product.

According to some embodiments, a method of producing a plasma production product is provided. The method includes forming an electrolyte mixture comprising sodium chloride (NaCl) and a liquid, wherein the electrolyte mixture is housed by a bioreactor tank; and supplying electrical power to the electrolyte mixture via a first electrode at least partially surrounded by the electrolyte mixture and a second electrode at least partially surrounded by the electrolyte mixture to produce the plasma production product.

Unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Various operations of embodiments and/or examples are provided herein. The order in which some or all of the operations are described herein should not be construed as to imply that these operations are necessarily order dependent. Alternative ordering will be appreciated by one skilled in the art having the benefit of this description. Further, it will be understood that not all operations are necessarily present in each embodiment and/or example provided herein. Also, it will be understood that not all operations are necessary in some embodiments and/or examples.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed is:

1. A plasma production system comprising:
a bioreactor tank housing:
an electrolyte mixture comprising sodium chloride (NaCl) and a liquid;
a first electrode at least partially surrounded by the electrolyte mixture; and
a second electrode at least partially surrounded by the electrolyte mixture; and
a power supply electrically connected to the first electrode and the second electrode and configured to supply electrical power to the electrolyte mixture via the first electrode and the second electrode to produce a plasma activated product.

2. The plasma production system of claim 1, wherein the power supply comprises at least one of:
a Direct Current (DC) power supply configured to supply DC electrical power to the electrolyte mixture; or
an Alternating Current (AC) power supply configured to supply AC electrical power to the electrolyte mixture.

3. The plasma production system of claim 1, wherein the power supply is configured to:
supply first electrical power having a first voltage to the electrolyte mixture during a first period of time; and
supply second electrical power having a second voltage to the electrolyte mixture during a second period of time, wherein the second voltage is different than the first voltage.

4. The plasma production system of claim 1, wherein the power supply is configured to:
supply first electrical power having a first current to the electrolyte mixture during a first period of time; and
supply second electrical power having a second current to the electrolyte mixture during a second period of time, wherein the second current is different than the first current.

5. The plasma production system of claim 1, wherein the power supply is configured to:
supply first electrical power having a first frequency to the electrolyte mixture during a first period of time; and
supply second electrical power having a second frequency to the electrolyte mixture during a second period of time, wherein the second frequency is different than the first frequency.

6. The plasma production system of claim 1, wherein the power supply is configured to:
supply first electrical power having a first voltage, a first current and a first frequency to the electrolyte mixture during a first period of time; and
supply second electrical power having a second voltage, a second current and a second frequency to the electrolyte mixture during a second period of time, wherein at least two of:
the second voltage is different than the first voltage;
the second current is different than the first current; or
the second frequency is different than the first frequency.

7. The plasma production system of claim 1, wherein at least one of:
a ratio of the sodium chloride to the liquid by weight is between about 0.01:1 to about 0.2:1; or
a ratio of the sodium chloride to the liquid by weight is between about 0.05:1 to about 0.1:1.

8. The plasma production system of claim 1, wherein at least one of:
the liquid comprises distilled water; or
the plasma activated product comprises at least one of a cold atmospheric plasma or a biocompatible plasma.

9. The plasma production system of claim 1, wherein at least one of:
the electrical power supplied by the power supply has a current that is at least one of:
between about 20 milliamperes to about 1000 milliamperes; or
between about 88 milliamperes to about 215 milliamperes;
the electrical power supplied by the power supply has a voltage that is at least one of:
between about 1 millivolt to about 100 millivolts; or
between about 8.16 millivolts to about 17.88 millivolts; or
the electrical power supplied by the power supply has a frequency that is at least one of:
between about 1 megahertz to about 1000 megahertz; or
between about 10 megahertz to about 100 megahertz.

10. The plasma production system of claim 1, wherein the first electrode comprises at least one of:
copper (Cu);
gold (Au);
silver (Ag);
zinc (Zn);
iron (Fe); or
graphite (Gr).

11. The plasma production system of claim 1, wherein the second electrode comprises:
an electrode body; and
a coating covering at least a portion of the electrode body, wherein the coating is at least partially surrounded by the electrolyte mixture.

12. The plasma production system of claim 11, wherein:
the coating comprises at least one of:
a metal oxide layer; or
a graphite layer.

13. The plasma production system of claim 11, wherein:
the electrode body comprises at least one of:
a metal; or
a graphite (Gr).

14. The plasma production system of claim 13, wherein the metal comprises at least one of:
copper (Cu);
gold (Au);
silver (Ag);
zinc (Zn); or
iron (Fe).

15. The plasma production system of claim 1, wherein the second electrode comprises:
a copper (Cu) body; or
a copper oxide coating covering at least a portion of the copper body, wherein the copper oxide coating is at least partially surrounded by the electrolyte mixture.

16. The plasma production system of claim 1, comprising at least one of:
one or more first electrodes to which the first electrode is connected, wherein the one or more first electrodes comprise at least one of:
copper (Cu);
gold (Au);
silver (Ag);
zinc (Zn);
iron (Fe); or
graphite (Gr); or
one or more second electrodes to which the second electrode is connected, wherein the one or more second electrodes comprise at least one of:
copper (Cu);
gold (Au);
silver (Ag);
zinc (Zn);
iron (Fe); or
graphite (Gr).

17. A method of treating a patient, comprising:
treating the patient using the plasma activated product produced in claim 1, wherein the treating the patient comprises administering the plasma activated product to the patient at least one of via injection, via inhalation, via oral administration or via skin absorption.

18. The method of claim 17, wherein treating the patient comprises at least one of:
treating cancer using the plasma activated product;
treating diabetes using the plasma activated product;
regulating liver enzymes using the plasma activated product;
treating an infectious wound using the plasma activated product;
treating a genital wart using the plasma activated product;
treating an oral condition using the plasma activated product;
treating Scleroderma using the plasma activated product;
treating a foot ulcer using the plasma activated product;
treating a bed sore using the plasma activated product;
treating an infectious disease using the plasma activated product;
relieving pain using the plasma activated product;
treating an antibiotic-resistant virus using the plasma activated product;
treating bacteria using the plasma activated product;
treating a fungus using the plasma activated product;
treating a skin disease using the plasma activated product;
treating Epidermolysis Bullosa (EB) using the plasma activated product;
treating sinusitis using the plasma activated product;
treating ear infection using the plasma activated product;
treating an eye infection using the plasma activated product; or
treating a bone infection using the plasma activated product.

19. A method of using the plasma activated product produced in claim 1, comprising at least one of:
applying a non-alcoholic disinfectant comprising the plasma activated product;
applying a sterilizer comprising the plasma activated product;
applying an agricultural reinforcement fertilizer comprising the plasma activated product; or
applying a pesticide comprising the plasma activated product.

20. A method of producing a plasma activated product comprising:
forming an electrolyte mixture comprising sodium chloride (NaCl) and a liquid, wherein the electrolyte mixture is housed by a bioreactor tank; and
supplying electrical power to the electrolyte mixture via a first electrode at least partially surrounded by the electrolyte mixture and a second electrode at least partially surrounded by the electrolyte mixture to produce the plasma activated product.
